(19) United States
(12) Patent Application Publication
(10) Pub. No.: US 2006/0172951 A1
(43) Pub. Date: Aug. 3, 2006

(54) Age related macular degeneration and cutaneous signs of mercury toxicity

(76) Inventor: Paul I. Dantzig, New York, NY (US)

Correspondence Address: BERNARD MALINA, ESQ.;MALINA & ASSOCIATES, P.C., Suite 501, 60 East 42nd Street, NEW YORK, NY, 10165, US

(21) Appl. No.: 11/333,049

(22) Filed: Jan. 17, 2006

Related U.S. Application Data
(60) Provisional application No. 60/648,866, filed on Jan. 28, 2005.

(51) Int. Cl. A61K 38/05; A61K 31/198; A61K 31/195; A61K 31/13; A61K 31/19

(52) U.S. Cl. 514/18; 514/562; 514/557; 514/665

(57) ABSTRACT

A method is provided for treating age related macular degeneration and Grover's disease by administering a safe and effective amount of a chelation agent to a person who would benefit from such treatment. Of particular effectiveness are chelation agents with sulfur atoms and which through the sulfur bind to mercury. A particularly effective agent is 2,3-dimercaptosuccinic acid or salt thereof

This application claims priority from U.S. Provisional Patent Application No. 60/648,866 entitled “Age Related Macular Degeneration and Cutaneous Signs of Mercury Toxicity” filed Jan. 28, 2005.

### BACKGROUND OF THE INVENTION

1. Field of the Invention

The invention concerns a relationship of mercury toxicity to macular degeneration and Grover's disease and a method for treating these medical conditions.

2. The Related Art

Age related macular degeneration is the leading cause of blindness in the world. It is estimated that one hundred million people are afflicted with the disease. Over the past three decades, the disease has increased dramatically and now accounts for vision loss in as many as 18% of the population over the age of 85. It is rarely seen in people under 50 and is more common in women and people who smoke. It is predicted that the afflicted will quadruple in the next ten years. The disease is devastating because it progressively destroys central vision thus making it impossible to perform daily functions such as reading, driving and recreation. The cause is unknown. Treatment is only supportive with some minor improvement noted in recent modalities including laser and photodynamic therapy. Antioxidants and nutritional supplements have been used with varying degrees of success.

Grover's disease (transient acantholytic dermatosis) is a chronic cutaneous eruption also with an unknown etiology. Like macular degeneration, the incidence has increased over the past three decades and distribution is more common in industrialized nations. It is also predominantly in people over 50 but has been reported to be more common in men. Pruritus can be mild to severe and the disease can last for years. Treatment is frequently symptomatic and the disease is often resistant to therapy, including oral and tropical steroids, isotrentinoin, and phototherapy.

Recently, a new cutaneous sign of mercury toxicity was described by Dantzig in J. Am. Acad. Dermatol., December 2003, 1109-1111. This sign consists of a generalized or localized erythematous popular eruption with only mild pruritus and is usually seen in people under the age of 50.

An object of the present invention is to provide a method for treating age related macular degeneration and Grover's disease.

### SUMMARY OF THE INVENTION

A method is provided for treating age-related macular degeneration by administering to a person who would benefit from such treatment a safe and effective amount of a chelation agent.

A method is also provided for treating Grover's disease by administering to a person who would benefit from such treatment a safe and effective amount of a chelation agent.

### DETAILED DESCRIPTION OF THE INVENTION

Mercury pollution is becoming a major ecological problem due to the contamination of water and seafood. Mercury is present in coal and oil and when these are burned, it vaporizes, eventually falling with precipitation into water and farmland. Atmospheric mercury also occurs from the inappropriate disposal of industrial products including medical waste, lights, computers and thermostats. In water, mercury is picked up by lower forms of life where it is methylated (its most toxic form) and works its way up the food chain to larger fish and humans. Mercury is extremely toxic with an affinity for neural tissue. It combines with the sulfide bonds in mitochondria, robbing the cell of energy and causing cell death. Since mercury deposits itself intracellularly, only a small amount is free in the serum and filterable through the kidneys. Much of the mercury is excreted through the intestines and is therefore recycled, which accounts for its half life in the body of at least two months. Because of this, only small amounts of mercury consumption can lead to a residual reservoir in the body. However, there is a paucity of studies linking low levels of mercury toxicity to any diseases. Most studies center around acute cases of mercury poisoning and therefore it is believed that blood levels under 15 or 20 u/Liter are safe.

Now it has been shown that very low levels of mercury are found in patients with Grover's disease and age related macular degeneration. Also, the mercury blood levels in most cases correlated with the severity of symptoms and the number of lesions. Electron microscopic studies of Grover's disease have shown a loss of intercellular and intracellular proteins, suggesting an interference with cell metabolism and protein synthesis. This would be consistent with the toxic effects of mercury. Electron microscopy in macular degeneration likewise shows an abnormal protein patter.

Grover's disease may represent a cutaneous marker for age related macular degeneration and may share common etiology with it. Both the skin and retina are separated by a basement membrane which with age degenerates, creating gaps and possibly allowing the crossing of toxins such as mercury.

Accordingly, the present invention concerns a treatment for age related macular degeneration and Grover's disease. These conditions can be treated by administering chelation agents which are believed to complex mercury and remove the resultant complex from the body or at least cause mercury inactivation. Non-limiting examples of chelation agents include citric acid, phosphates, ethylenediaminetetraacetic acid (EDTA) salts such as disodium and tetrasodium EDTA, (4-amino-1-hydroxy butylidene) diphosphonic acid salt (available as the monosodium salt trihydrate under the name alendronate sodium, and [1-hydroxy-2(3-pyridinyl)ethylene]bis[phosphonic acid]salt available as the monosodium salt in risedronate sodium pentahydrate. Particularly useful chelation agents for the present invention are those which include sulfur atoms, especially where the sulfur atoms are directly involved in the chelation of mercury. Illustrative examples include 2,3-dimercaptosuccinic acid or salt thereof (commercially available as Succimer), beta, beta-dimethylcysteine (known as Penicillamine); mercaptoethylamine (also known as dimercaptol); 2,3-dimercapto-1-propanol (known as dimercaprol), N-acetylcysteine; and glutathione. Most preferred is Succimer.

The chelation agents can prevent, inhibit, retard, cure or any combination of these effects relative to age related macular degeneration or Grover's disease. Administration of the chelation agent preferably is done orally through ingestion of a suitable pill with active agent. However, administration can also be accomplished through intravenous injection or topical application. Oral administration can be by tablet, liquid or gel caplet formats. The active agent will normally be delivered in a formula having an inert carrier. Suitable carriers include sugars, inorganic powders, water, thickening agents (e.g., gums) and combinations thereof.

In one aspect of the present invention, the active chelation agent is suspended or dissolved in a carrier or formulated into a multiplicity of unit dose tablets or caplets or as a liquid in a suitable container. Containers for the formulated active will be provided with child-proof closures. In conjunction with packaging, the containers will have labels and/or inserted printed instructions advising the person requiring treatment on how best to administer the chelation agent for treatment of Grover's disease or macular degeneration.

For a better understanding of the present invention, specific embodiments of the invention are presented below. The example and illustrations are not to be construed in any way as limiting the scope of the invention. In this specification, parts, percentages and ratios should be considered being by weight unless otherwise indicated.

### EXAMPLE

A study was conducted to evaluate the postulate that Grover's disease may represent a variant of mercury eruption in older people. Further, the study was intended to evaluate the relationship between macular degeneration and cutaneous signs of mercury toxicity.

Materials and Methods

Fourteen patients with age related macular degeneration (ages 57-97) were randomly recruited via radio advertisements. All patients were diagnosed with macular degeneration by an ophthalmologist using the following criteria: (1) blurred central vision; (2) positive test with Amsler grid; (3) visual macular retinopathy including signs of Drusen formation. All fourteen patients gave verbal consent for physical exam of chest, abdomen and back, a shave biopsy of a cutaneous lesion and blood test for mercury. Seven were women and seven were men.

Fourteen patients with Grover's disease (ages 55-85) were randomly selected from a private dermatology practice. All patients were diagnosed using the following criteria: (1) complaints of pruritic eruption of the chest, abdomen or back; (2) physical exam showing discreet small (<2 mm) scattered erythematous scaly or crusted papules of chest, abdomen or back; (3) biopsy showing a superficial perivascular infiltrate of lymphocytes, suprabasal clefts and acantholytic cells. All patients gave verbal consent for a shave biopsy of a skin lesion and a blood test for mercury. Two were women and twelve were men.

Two control groups were randomly selected. Group 1 consisted of fourteen patients without any signs or symptoms of Grover's disease or macular degeneration. There were four women and ten men and ages ranged from 52-93. A random blood mercury was drawn on each patient and compared with the fourteen patients with Grover's disease.

Control Group 2 consisted of nine patients with detectable blood mercury but no signs of macular degeneration. There were two women and seven men and ages ranged from 56-93. These patients were examined for signs of Grover's disease and compared with the fourteen patients with macular degeneration.

Blood mercury levels were measured in all patients by inductively coupled plasma mass spectrometry by Nichols Laboratory.

Analysis of the results from each patient is recorded in Table I and a comparison of the diseased patients to that of controls is recorded in Table II.

TABLE I
Patients With Macular Degeneration and
Cutaneous Signs of Mercury Toxicity

| | | | | Blood | Approx. No. of |
|---|---|---|---|---|---|
| Patient | Age | Sex | Skin Sign | Mercury | Cutaneous Lesion |
| 1 | 57 | M | Spongiotic papule | 5 | 2-4 |
| 2 | 68 | M | Grover's | 3 | 2-4 |
| 3 | 72 | F | Grover's | 7 | 2 |
| 4 | 78 | M | Grover's | 8 | 10-20 |
| 5 | 70 | F | Grover's | 10 | 5-8 |
| 6 | 82 | F | Grover's | 0 | 2 |
| 7 | 60 | M | Spongiotic papule | 8 | 5-8 |
| 8 | 97 | M | Grover's | 3 | 2-4 |
| 9 | 79 | F | Grover's | 1.3 | 2.4 |
| 10 | 64 | M | Grover's | 1.7 | 2.4 |
| 11 | 82 | M | Grover's | 3 | 5-8 |
| 12 | 78 | F | Grover's | 3 | 5-8 |
| 13 | 83 | F | None on Biopsy | 3 | 2-4 |
| 14 | 60 | F | Grover's | 10 | 5-8 |

TABLE II
Comparison of Patients with Macular Degeneration
and Grover's Disease to control

| | | | Skin | Skin Signs |
|---|---|---|---|---|
| | | Mean Blood | Symptoms | (No. of |
| | | Mercury | Pruritus | Papules) |
| 1. | Patients with macular | 4.5 | None | <10 |
| | Degeneration and | (13/14) | | |
| | Grover's Disease | | | |
| 2. | Patients with detectable | 12 | None | None |
| | blood mercury and no | (9/9) | | |
| | signs of macular | | | |
| | degeneration | | | |
| | (Control Group I) | | | |
| 3. | Patients with Grover's | 10 | moderate | Many |
| | Disease | (14/14) | to severe | |
| 4. | Patients without macular | 2 | None | None |
| | degeneration or Grover's | (2/14) | | |
| | disease (Control Group 2) | | | |

Results

Of the fourteen patients with macular degeneration, all patients had at least one erythematous papule or crusted papule of the chest, abdomen or back but no patient had more than twenty. None of the patients complained of pruritus or was aware of any skin problem. A skin biopsy of each patient revealed Grover's disease (acantholytic epidermal cells) in eleven patients, a spongiotic papule as described by Dantzig in two patients and a nonspecific biopsy in one patient (Note: since this patient had more than one lesion, physician error in diagnosing and biopsing the wrong lesion cannot be ruled out). Thirteen of the fourteen patients had measurable blood mercury (mean 4.5 u/Liter). The one patient who had a negative reading still gave of history of fish consumption (the major source of methyl mercury exposure) so an error in quantitative analysis cannot be ruled out (Table I).

Of the nine control patients with measurable blood mercury levels (mean—12 u/Liter) and no macular degeneration, none showed signs of Grover's disease on physical examination.

Of the fourteen patients with Grover's disease, all had measurable blood mercury (mean—10 u/Liter). All patients complained of pruritus and were aware of skin lesions. All fourteen patients were treated with topical steroids without improvement. However, three patients were treated with diet (no seafood) and chelation with Succimer (200-300 mg TID×1-2 months) with clearing of the eruption, thus lending credence to mercury as the cause of the disease.

Of the fourteen patients in the control group for Grover's disease, twelve had no detectable blood mercury and two had blood mercury levels of 10 and 16 micrograms/Liter.

These examples reveal that Grover's disease may be a marker for age related macular degeneration. Further, for the first time, it is shown that a specific toxin, mercury, may play an etiological role in the development of age related macular degeneration. Quite importantly, the clinical study has shown that use of chelation treatment such as with Succimer, have a beneficial effect in the treatment of Grover's disease and age related macular degeneration.

Now having set forth the general nature and at least one example of the present invention, the scope of the invention is hereinafter set forth in greater particularity in the appended claims.

What is claimed is:

1. A method for treating macular degeneration comprising administering to a person who would benefit from such treatment a safe and effective amount of a chelation agent.

2. The method according to claim 1 wherein the chelation agent comprises a compound with at least one sulfur atom.

3. The method according to claim 2 wherein the chelation agent through the at least one sulfur atom binds with mercury.

4. The method according to claim 1 wherein the chelation agent is a 2,3-dimercaptosuccinic acid or salt thereof.

5. The method according to claim 1 wherein the chelation agent is selected from the group consisting of beta,beta-dimethylcysteine, mercaptoethylamine, 2,3-dimercapto-1-propanol, N-acetylcysteine and glutathione.

6. A method for treating Grover's disease comprising administering to a person who would benefit from such treatment a safe and effective amount of a chelation agent.

7. The method according to claim 5 wherein the chelation agent comprises a compound with at least one sulfur atom.

8. The method according to claim 7 wherein the chelation agent through the at least one sulfur atom binds with mercury.

9. The method according to claim 6 wherein the chelation agent is a 2,3-dimercaptosuccinic acid or salt thereof.

10. The method according to claim 6 wherein the chelation agent is selected from the group consisting of beta,beta-dimethylcysteine, mercaptoethylamine, 2,3-dimercapto-1-propanol, N-acetylcysteine and glutathione.
